(19) United States
(12) Patent Application Publication
(10) Pub. No.: US 2017/0323414 A1
(43) Pub. Date: Nov. 9, 2017

(54) METHOD OF DECREASING THE TIME FOR OBTAINING DIAGNOSTIC DNA TEST RESULTS AND KIT FOR SAME

(71) Applicant: Stacey Watt, Grand Island, NY (US)

(72) Inventor: Stacey Watt, Grand Island, NY (US)

(21) Appl. No.: 15/586,842

(22) Filed: May 4, 2017

Related U.S. Application Data
(60) Provisional application No. 62/333,307, filed on May 9, 2016.

(51) Int. Cl. G06Q 90/00; B65D 81/113; B65B 7/16; B65D 27/00; C12Q 1/68; B65D 85/00

(52) U.S. Cl. CPC G06Q 90/00; C12Q 1/6806; B65D 85/70; B65B 7/16; B65D 27/00; B65D 81/113

(57) ABSTRACT

The present invention is directed to a method of increasing the speed at which diagnostic DNA test results are provided and a kit to streamline the process and paperwork of the method. The kit includes all of the components required by the diagnostic DNA testing lab in order for them to perform the diagnostic DNA test.

### CROSS-REFERENCE TO RELATED APPLICATIONS

This application claims the benefit of, and incorporates by reference, U.S. Provisional Application No. 62/333,307, filed May 9, 2016.

### FIELD OF INVENTION

This invention is directed to a method of increasing the efficiency and decreasing the speed at which diagnostic DNA test results are provided and a kit to streamline the process and paperwork of same.

### BACKGROUND OF INVENTION

Malignant hyperthermia (“MH”) is a rare, but life-threatening, disease that is triggered by certain anesthetic drugs (or triggering agents), including succinylcholine and general anesthetics. An episode of malignant hyperthermia may include some or all of the following symptoms: a fast rise in body temperature (105° F. or higher), severe muscle contractions, bleeding, tachycardia, hyperthermia, acidosis, dark brown urine, muscle ache, muscle rigidity, and muscle stiffness.

MH is not particularly common, occurring in 1 in 5,000-50,000 instances in which individuals are given a triggering agent. Triggering agents include: inhaled general anesthetics; desflurane; enflurane; ether; halothane; isoflurane; methoxyflurane; sevoflurane; and succinylcholine. Nonetheless, MH is particularly problematic because in many cases it isn't diagnosed until the patient has had an MH episode as the result of exposure to one of the trigger agents during anesthesia. After a presumed episode of MH, it is very important to confirm that the patient actually does have MH.

Most people with MH are not aware that they have the disease. Because most patients don't know they have MH, an anesthesiologist will only learn that a patient has the disease when an MH episode occurs as a result of exposing the patient to one of the triggering agents. As can be imagined, it is beneficial for the anesthesiologist and the patient to know about the presence of MH in advance through a diagnostic test. If the patient has MH, he or she can wear a medic alert bracelet so that the anesthesiologist can avoid using triggering agents during anesthesia. Moreover, because MH is hereditary, this information is helpful for the patient's family members. If the patient's family members learn that the patient has MH, then they know there is a chance that they also have MH and they can also get tested for the presence of MH.

Currently, the process of diagnosing MH is inefficient. As a result, there is a significant delay between the time of obtaining the sample from the patient and diagnosis of the disease. This is because of the piecemeal steps and paperwork required by the testing lab. To begin the testing process, the physician or hospital fills out a paper form with information, sends it to the lab, then the lab responds by requiring another paper form with additional information. The extensive back and forth between the physician or hospital and the lab becomes burdensome and very time consuming and causes a lot of delays.

Diagnosis of MH requires either a muscle biopsy or a DNA sample. In the case of the muscle biopsy, the test performed is called contracture. For this test, a 3-5 cm long and 1-1.5 cm in diameter muscle sample is taken from the patient on an outpatient basis. The muscle sample is then exposed to halothane (a known triggering agent for MH). If the muscle is susceptible to MH, then it will contract more frequently than an unsusceptible muscle and will develop a sustained force generation known as a contracture. This type of test is approximately 95% accurate for diagnosing MH. Because taking a muscle biopsy from a patient is uncomfortable and invasive, other diagnostic tests have been developed, in particular, DNA testing for genetic markers of MH.

In the case of a DNA sample, genetic testing is used to ascertain whether the person has the genetic markers that indicate the presence of MH. MH is inherited as an autosomally dominant trait with variable expression and incomplete penetrance. Recently, a mutation in the RYR1 gene has been identified as contributing to the presence of MH. Additionally, other gene markers are being identified and in the future these markers may be used to diagnose MH. Thus, MH is a genetically diagnosable disease.

When an MH episode occurs after patient exposure to a triggering agent, an emergency situation develops. As mentioned above, an MH episode is life threatening. Thus, the response needs to be immediate. A drug called dantrolene is administered to the patient to counter the MH symptoms. MH episodes involve an increased release of calcium in the system, which causes a rise in body temperature and rigidity of muscles. The dantrolene blocks the calcium from being further released and ultimately drops the patient's temperature and relaxes the muscles. During an MH crisis, the patient's blood chemistry is constantly monitored.

In some cases, there are other events that look like an MH episode, including a sepsis reaction or an overdose of illicit drugs such as amphetamines. Dantrolene will help in these cases as well, but ultimately, these other events require different medical responses and it is important to ascertain whether the event was actually an MH episode or something else. This is the case because of the heritability of MH as discussed above. It is important for the patient's family to know whether they should be tested for MH or not.

At the time of a presumed MH episode, a doctor will want to take a DNA sample and send it to the lab for genetic testing. As mentioned above, it is important to know whether the episode was an MH episode or something else because if the genetic markers are present in that patient, the patient's family can also be tested and if those individuals also have MH genetic markers, they can start wearing a medic alert bracelet so that a physician will know to avoid the triggering agents when treating that patient. The triggering agents are very efficient and effective, but there are alternatives available.

Currently, there is a huge time delay from the time of the MH episode to the actual DNA testing and test results. This is due to all of the piecemeal steps and paperwork involved in genetic MH testing. Much of the paperwork involves back and forth between the doctor or office and the testing lab.

### SUMMARY OF THE INVENTION

Accordingly, it is the objective of this invention to streamline the process and paperwork for obtaining a positive or negative MH diagnosis by providing a kit that can be used to significantly reduce the amount of time required for obtaining a diagnostic DNA test result. As mentioned above, when a presumed MH episode occurs, the physician can obtain a sample and use the kit of the present disclosure to assemble everything that is required by the testing lab.

The problem of delay and inefficiency in diagnostic DNA testing is solved by streamlining the process and paperwork required by the lab for DNA testing.

### BRIEF DESCRIPTION OF THE DRAWINGS

FIG. 1 depicts a kit of the present disclosure.

### DETAILED DESCRIPTION OF THE INVENTION

### Parts List

Kit 10
Box 12
Cover 14
Box Seal 15
Pocket 16
Sides 18
Bottom 20
Foam padding 22
Indentations 24
Collection Vessel 26
Collection Vessel Fill Line 26a
Collection Vessel Seal 27
Paperwork 28
Instruction Sheet 30
Envelope 32
Patient Information Form 34
Completed Patient Information Form 34a
Informed Consent Form 36
Completed Informed Consent Form 36a
Sticker 38
Address Label 40
Postage Label 41
Medical History Form 42
Completed Medical History Form 42a
Waiver Form 44
Completed Waiver Form 44a
Requisition Form 46
Completed Requisition Form 46a
Return Box seal 48

One method of streamlining the process and paperwork for obtaining diagnostic DNA testing results, includes the step of providing an easily accessible kit 10 for a physician to utilize at the location of obtaining a blood sample, saliva sample, or another DNA sample. In particular, this kit 10 can be easily accessible during administration of an MH triggering agent in the event that an MH episode occurs. If a suspected MH episode occurs, then the physician can use the kit 10 immediately to obtain a blood or saliva sample for purposes of diagnostic DNA testing to determine whether the patient has MH. Of note, the kit 10 includes a box 12 for containing all of the items necessary to send to the genetic testing laboratory.

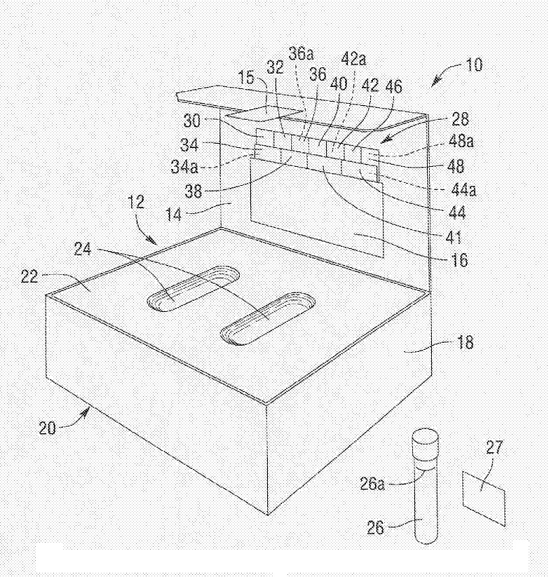

Turning now to FIG. 1, a kit 10 of the present disclosure is depicted, wherein the kit 10 includes a box 12 having a cover 14, wherein the cover 14 has a box seal 15 used to seal the box 12 before it is used. The cover 14 also contains a pocket 16. The box 12 also has sides 18 and a bottom 20, wherein the bottom 20 and sides 18 contain foam padding 22, wherein the foam padding 22 defines indentations 24 sized for receiving a collection vessel 26 (of which there can be more than one). Collection vessel 26 has fill line 26a and is sealed with collection vessel seal 27. Pocket 16 contains paperwork 28.

Paperwork 28 includes at least one of the following: instruction sheet 30; envelope 32; patient information form 34: informed consent form 36; sticker 38; address label 40; postage label 41; medical history form 42; waiver form 44; requisition form 46; and return box seal 48, and in one embodiment, paperwork 28 includes all of the above.

In a preferred embodiment, the instruction sheet 30 has instructions for how to use the kit 10 and how to complete the rest of the forms of paperwork 28. Preferably, envelope 32 is large enough to contain box 12 so that once the DNA sample is obtained and deposited into the collection vessel 26, collection vessel 26 can be sealed with collection vessel seal 27, placed back into indentations 24, and sent to a genetic testing lab (not shown). Patient information form 34 allows the physician to input the patient's insurance information and other relevant information required by the genetic testing lab. Informed consent form 36 provides the patient with all of the known risks of the testing procedure and the patient signs this form after reviewing everything. Sticker 38 can be used to write or print the patient's name thereon. Address label 40 contains the address for the designated genetic testing lab and postage label 41 has prepaid postage thereon and both are preferably placed on envelope 32 prior to mailing.

In a preferred embodiment, the kit 10 has two indentations 24 for receiving two collection vessels 26. The collection vessels 26 may be blood tubing or two sets of salivary tubing.

In a preferred embodiment, the pocket 16 is comprised of a clear plastic panel that forms the pocket 16. In another embodiment, the pocket 16 may be made of another material such as cardboard paper or paper.

In a preferred embodiment, the paperwork 28 contains patient information form 34, informed consent form 36, medical history form 42, and waiver form 44 wherein these forms include checkboxes so that the physician can save time by checking the appropriate checkbox rather than having to fill in all of the information by hand. In another preferred embodiment, the kit 10 contains two sets of the paperwork 28 so that one copy is easily and quickly retained for the patient's records. In another embodiment, the instruction form 30 includes all of the steps the physician must take in order to complete the kit 10 and return it to the lab. In another embodiment, the instruction sheet 30 is laminated.

A method of using the kit 10 is also disclosed including the steps of: opening a kit 10 for diagnostic DNA testing by unsealing box seal 15 from box 12, wherein the box 12 contains a collection vessel 26 for receiving a DNA sample, collection vessel holder 24, collection vessel seal 27, and paperwork 28. Paperwork 28 further includes an instruction sheet 30, envelope 32, patient information form 34, informed consent form 36, sticker 38, address label 40, postage label 41, medical history form 42, waiver form 44, requisition form 46, and return box seal 48.

The method further comprises removing the paperwork 28 from box 12 and completing or filling in patient information form 34, thereby forming a completed patient information form 34a (shown in dashed lines), completing informed consent form 36, thereby forming completed informed consent form 36a (shown in dashed lines), completing medical history form 42, thereby forming completed medical history form 42a (shown in dashed lines), completing waiver form 44, thereby forming completed waiver form 44a (shown in dashed lines), and completing requisition form 48, thereby forming completed requisition form 48a (shown in dashed lines).

In a preferred embodiment, completed patient information form 34a is sent to the diagnostic DNA testing lab so that they can bill the insurance company. Preferably, the completed informed consent form 36a, the completed medical history form 42a, and completed waiver form 44a, are kept by the physician with the patient's medical records.

In a preferred embodiment, the requisition form 46 is completed prior to the step of obtaining a saliva sample, blood sample, or other DNA sample. In another preferred embodiment, the requisition form 46 is a letter of necessity. In another preferred embodiment, the kit includes an informed consent form 36. Finally, in another preferred embodiment, the kit contains both a letter of necessity 46 and an informed consent 36. The letter of necessity 46 can be sent to an insurance company at the time of testing so that the insurance company will agree to pay for the diagnostic DNA test. The informed consent form 36 explains the risks of the testing to the patient and requires them to authorize the testing.

The method further includes the step of obtaining a blood sample, saliva sample, or other DNA sample from a patient.

In the embodiment wherein the sample is blood, the blood is drawn directly into the collection vessel 26, wherein the collection vessel 26 is a test tube containing an anti-coagulant and the method further includes the step of shaking the test tube so as to mix the blood sample with anticoagulant.

In the embodiment wherein the sample is saliva, the saliva may be collected by a dry procedure, wet procedure, or non-invasive procedure. The dry procedure requires a cytobrush, buccal swab or other collection device to be inserted into the mouth to scrape tissue from the gum or cheek and the cytobrush, buccal swab or other collection device is placed in the collection vessel. The wet procedure requires swishing liquids in the mouth and spitting them into the collection vessel 26. The non-invasive procedure requires spitting into the collection vessel 26 and adding a solution to stabilize the DNA. In this embodiment, the collection vessel further 26 comprises at least one of: a cytobrush, a buccal swab, another collection device, a liquid, and stabilizing solution.

The method further includes the step of placing the collection vessels 26 into the indentations 24 in the foam padding 22 of the bottom 20 of box 12, optionally placing the completed patient information form 34a in the pocket 16 of box 12, and sealing the box 12 with the return box seal 38. The method further includes the step of: enclosing the sealed box 12 in the envelope 32, placing address label 40 and postage label 41 on envelope 32, and mailing the envelope 32 and box 12 to a DNA genetic testing facility.

In another preferred embodiment, the collection vessel or test tube (optionally with anti-coagulant) is marked to show the minimum amount of sample required. In another preferred embodiment, the collection vessel 26 or test tube is filled to at least the collection vessel fill mark 26a, which indicating the minimum amount of sample required.

In another preferred embodiment, a patient copy of some of the paperwork 28 is provided to the patient or patient's family. In another embodiment, the sticker 38 is used to identify the patient on some of the paperwork 28.

Some advantages of the present disclosure include a significant decrease in the amount of time it takes for the diagnostic DNA test results to return. Typically, it takes several weeks for everything to be completed because of the piecemeal steps and paperwork. By using the kit 10 of the present disclosure, there is significantly less back and forth as all of the steps are completed at the same time and provided to the lab in one efficient kit 10. This also provides the advantage of saving physicians and staff time.

It is envisaged that the kit will be beneficial to hospitals, physicians offices, and emergency centers. Essentially, it would be beneficial for any place that does surgery or may administer an MH triggering agent to a patient to have the kit of the present disclosure easily accessible.

It is to be understood that the above-described kit 10 is capable of being used to detect any other genetically diagnosable diseases without limitation. For example, many cancers and other diseases can be genetically diagnosable.

It will be appreciated by those skilled in the art that while the Method of Decreasing the Time for Obtaining Diagnostic DNA Test Results and Kit for Same has been described in detail herein, the invention is not necessarily so limited and other examples, embodiments, uses, modifications, and departures from the embodiments, examples, uses, and modifications may be made without departing from the process and all such embodiments are intended to be within the scope and spirit of the appended claims.

What is claimed is:

1. A kit for decreasing the time for obtaining a diagnostic DNA test result, the kit comprised of:
a box including a cover having a pocket, sides, and a bottom containing foam padding, wherein the foam padding defines indentations sized to receive a collection vessel;
a collection vessel disposed in the indentations in the foam padding;
a collection vessel seal; and
paperwork disposed in the pocket.

2. A kit according to claim 1, wherein the pocket is made of plastic and the paperwork further includes:
an instruction sheet, an envelope, a patient information form, an informed consent form, a sticker, an address label; a postage label; a medical history form; a waiver form; a requisition form;
and a return box seal.

3. A kit according to claim 1, wherein the collection vessel is a tube suitable for collecting a blood sample.

4. A kit according to claim 1, wherein the collection vessel is a tube suitable for collecting a saliva sample.

5. A method for making a kit for decreasing the time for obtaining a diagnostic DNA test result, the method comprising the steps of:
providing a box including a cover having a pocket, sides, and a bottom containing foam padding, wherein the foam padding defines indentations sized to receive a collection vessel;
providing a collection vessel disposed in the indentations in the foam padding;
providing a collection vessel seal; and
providing paperwork disposed in the pocket.

6. A method according to claim 5, further comprising the step of providing additional paperwork including:
an instruction sheet, an envelope, a patient information form, an informed consent form, a sticker, an address label; a postage label; a medical history form; a waiver form; a requisition form; and a return box seal.

7. A method according to claim 5, further comprising the step of providing the collection vessel to be in the form of a tube suitable for collecting a blood sample.

8. A method according to claim 5, further comprising the step of providing a collection vessel to be in the form of a tube suitable for collecting a saliva sample.

9. A method of using a kit for decreasing the time for obtaining a diagnostic DNA test result, the method comprising the steps of:
opening a kit by breaking a seal on a box, wherein the box includes a cover having a pocket having paperwork disposed therein, sides, and a bottom containing foam padding, wherein the foam padding defines indentations sized to receive a collection vessel; and
removing paperwork disposed in the pocket.

10. The method according to claim 9, wherein the paperwork further includes: an instruction sheet, an envelope, a patient information form, an informed consent form, a sticker, an address label; a postage label; a medical history form; a waiver form; a requisition form; and a return box seal; and further comprising the step of reviewing the instruction sheet.

11. The method according to claim 10, further comprising the steps of completing the patient information form, completing the informed consent form, completing the medical history form, completing the waiver form, and completing the requisition form, thereby creating a completed insurance form, a completed informed consent form, a completed medical history form, a completed waiver form, and a completed requisition form.

12. The method according to claim 11, wherein the kit further includes a collection vessel disposed in the indentations in the foam padding and a collection vessel seal and further comprising the step of removing the collection vessel.

13. The method according to claim 12, further comprising the steps of:
obtaining a blood sample from a patient;
depositing the blood sample into the collection vessel, wherein the collection vessel further includes an anticoagulant;
shaking the collection vessel in order to mix the blood sample and the anticoagulant;
sealing the collection vessel with the collection vessel seal; and
placing the sealed collection vessel into the collection vessel receptacle of the box.

14. The method according to claim 12, wherein the collection vessel further includes a cytobrush, a buccal swab, or other collection device to obtain a saliva sample and further comprising the steps of:
using the cytobrush, buccal swab, or other collection device to obtain a saliva sample from a patient;
depositing the saliva sample into the collection vessel;
sealing the collection vessel with the collection vessel seal; and
placing the sealed collection vessel into the collection vessel receptacle of the box.

15. The method according to claim 12, wherein the kit further includes a liquid DNA stabilizing solution and further comprising the steps of:
obtaining a saliva sample from a patient, wherein the patient is provided the liquid DNA stabilizing solution to swish in the mouth;
depositing the saliva sample mixed with liquid DNA stabilizing solution into the collection vessel;
sealing the collection vessel with the collection vessel seal; and
placing the sealed collection vessel into the collection vessel receptacle of the box.

16. The method according to claim 12, wherein the kit further includes a liquid DNA stabilizing solution and further comprising the steps of:
obtaining a saliva sample or other DNA sample from a patient;
depositing the saliva sample or other DNA sample into the collection vessel and adding the liquid DNA stabilizing solution to the collection vessel;
sealing the collection vessel with the collection vessel seal; and
placing the sealed collection vessel into the collection vessel receptacle of the box.

17. The method according to claim 13, further comprising the steps of:
placing the completed patient information form into the pocket;
sealing the box with the return box seal;
placing the sealed box into the envelope;
placing the address label and the postage label onto the envelope; and
mailing the envelope.

18. The method according to claim 14, further comprising the steps of:
placing the completed patient information form into the pocket;
sealing the box with the return box seal;
placing the sealed box into the envelope;
placing the address label and the postage label onto the envelope; and
mailing the envelope.

19. The method according to claim 15, further comprising the steps of:
placing the completed patient information form into the pocket;
sealing the box with the return box seal;
placing the sealed box into the envelope;
placing the address label and the postage label onto the envelope; and
mailing the envelope.

20. The method according to claim 16, further comprising the steps of:
placing the completed patient information form into the pocket;
sealing the box with the return box seal;
placing the sealed box into the envelope;
placing the address label and the postage label onto the envelope; and
mailing the envelope.
